(19) United States
(12) Patent Application Publication
(10) Pub. No.: US 2019/0151280 A1
(43) Pub. Date: May 23, 2019

(54) Cannabinoid Compositions

(71) Applicant: Tilray, Inc., Seattle, WA (US)

(72) Inventors: Caleb Joshua Eades, Gabriola (CA); Orion Lekos, Bellingham, WA (US); Giri Cheekati, Mississauga (CA)

(21) Appl. No.: 16/195,454

(22) Filed: Nov. 19, 2018

Related U.S. Application Data
(60) Provisional application No. 62/588,059, filed on Nov. 17, 2017.
(60) Provisional application No. 62/654,815, filed on Apr. 9, 2018.

(51) Int. Cl. A61K 31/352; A61K 47/24; A61K 47/06; A61K 47/44; A61K 47/22; A61K 47/10

(52) U.S. Cl. CPC A61K 31/352; A61K 47/24; A61K 47/10; A61K 47/44; A61K 47/22; A61K 47/06

(57) ABSTRACT

Compositions for administering cannabinoid, including edible forms of cannabinoids. Phospholipids and waxes may be used to control the onset timing of cannabis drug effects.

### CROSS-REFERENCE TO RELATED APPLICATIONS

This application claims the benefit of U.S. Provisional Patent Application No. 62/588,059, filed on Nov. 17, 2017, and U.S. Provisional Patent Application No. 62/654,815, filed on Apr. 9, 2018. The entire contents of those applications are incorporated herein by reference.

### BACKGROUND OF THE INVENTION

The active ingredients of cannabis, in the form of cannabinoids—a diverse chemical family of compounds that act on cannabinoid receptors in cells and alter neurotransmitter release in the brain—can be useful in the practice of medicine. For example, consumption of delta-9-tetrahydrocannabinol (Δ9-THC, also referred to herein as “THC”) may be used to treat anorexia in people with HIV/AIDS, or nausea and vomiting in people undergoing chemotherapy.

Cannabis may be administered in a number of ways, including by smoking, vaporizing, by application of transdermal patches, and by consuming edible foods or products containing cannabis. Edible forms of cannabis are considered discreet and effective options for receiving the medicinal effects of cannabis without exposure to the potentially harmful risks of cannabis smoke. The onset timing of the drug effects of cannabis, however, may be markedly different depending on how it is administered. For example, there is a delayed onset of the drug effects when cannabis is ingested as compared to when cannabis extract is smoked. When consumers do not appreciate the longer time needed to experience drug effects when consuming cannabis in edible form, they may consume more than an intended amount of the drug, which can result in adverse effects.

To allow users to more safely and accurately administer edible cannabis, the time of onset of the drug may be controlled. The present inventions describe methods of preparation and chemistry compositions that allow greater control over drug effect onset time.

### SUMMARY OF THE INVENTION

It is an object of the present invention to provide improved compositions for administering cannabinoid. It is also an object of the present invention to control the onset timing of cannabis drug effects by including in the compositions, for example, phospholipids and waxes.

### BRIEF DESCRIPTION OF THE DRAWINGS

A further understanding of the invention can be obtained by reference to embodiments set forth in the illustrations of the accompanying drawings. Although the illustrated embodiments are merely examples for carrying out the invention, both the organization and method of operation of the invention, in general, together with further objectives and advantages thereof, may be more easily understood by reference to the drawings and the following description. Like reference numbers generally refer to like features (e.g., functionally similar and/or structurally similar elements).

The drawings are not intended to limit the scope of this invention, which is set forth with particularity in the claims as appended hereto or as subsequently amended, but merely to clarify and exemplify the invention.

FIG. 1 depicts the chemical structure of cannabidiol;

FIG. 2 depicts the chemical structure of delta-9-tetrahydrocannabinol;

FIG. 3 depicts the chemical structure of phosphatidylcholine.

### DETAILED DESCRIPTION OF THE INVENTION

The invention may be understood more readily by reference to the following detailed descriptions of embodiments of the invention. However, techniques, systems, and operating structures in accordance with the invention may be embodied in a wide variety of forms and modes, some of which may be quite different from those in the disclosed embodiments. Consequently, the specific structural and functional details disclosed herein are merely representative, yet in that regard, they are deemed to present embodiments for purposes of disclosure and to provide a basis for the claims herein, which define the scope of the invention. It must be noted that, as used in the specification and the appended claims, the singular forms “a”, “an”, and “the” include plural referents unless the context clearly indicates otherwise.

Generally, edible forms of cannabinoids are introduced through the gastrointestinal tract. Once in the gastrointestinal tract, the gut breaks down the edible form. The process of breaking down the edible form influences the time it takes for 49-THC to be absorbed into the bloodstream. Depending on the chemistry of the edible form, the drug can either quickly absorb into the bloodstream or take a long time to be digested and absorbed into the bloodstream.

Once in the bloodstream, the Δ9-THC travels via the portal vein to the liver, where it undergoes first-pass metabolism. Liver enzymes (primarily the cytochrome P450 system) hydroxylate Δ9-THC to form 11-hydroxytetrahydrocannabinol (11-OH-THC), a potent psychoactive metabolite that readily crosses the blood-brain barrier. See Mura P, Kintz P, Dumestre V, Raul S, Hauet T., THC Can be Detected in Brain While Absent in Blood, J. Anal. Toxicol. 2005, November-December; 29(8):842-3.

Ratio-specific phospholipids and waxes may be used to control the onset timing of cannabis drug effects. Phospholipids such as Phosphatidylcholine (PC) and Phosphatidylethanolamine (PH) may encapsulate the cannabinoid in a liposomal structure and quickly pass across the cell wall of the gut into the bloodstream. Phospholipids may thereby help to advance the onset timing of drug effects.

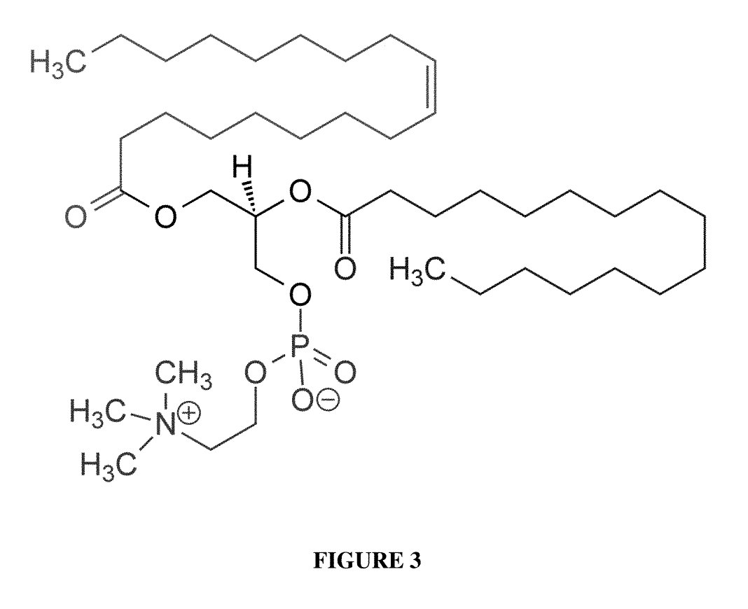

Cannabinoids may be solubilized with phospholipids in a solvent, such as ethanol, or with a lipid or terpene solution. The chemical structure of PC is depicted in FIG. 3. Phosphatidylethanolamines may be synthesized by the addition of cytidine diphosphate-ethanolamine to diglycerides, releasing cytidine monophosphate. S-Adenosyl methionine can subsequently methylate the amine of phosphatidylethanolamines to yield phosphatidylcholines.

The ratio of PC to PH may also influences the time to onset of drug effects. The ratio of PC to PH may be, for example, between the ranges of 60% PC to 40% PH and 100% PC to 0% PH. A phospholipid that is 90% to 100% PC has been found to provide an onset of cannabinoid drug effects in approximately five to 20 minutes. A ratio of 75% PC and 25% PH provides an onset of cannabinoid drug effects in approximately 30 to 40 minutes.

Waxes, such as beeswax, delay onset timing by binding the cannabinoids. Waxes are broken down slowly in the gut and do not release the cannabinoids until the wax is digested. A beeswax with medium chain triglyceride (MCT) oil can delay onset of drug effects by up to four hours.

Various methods of assessing time of onset were used to determine the effects of phospholipids and waxes. One method used to assess the impact on time of onset included measuring change in heart rate. On average, as onset of drug effects occurs, the heart rate increases by 20 beats per minute. Also, eye dilation and mood shifts were further used to detect onset of the drug effects.

The present invention is further illustrated, but not limited, with reference to the following examples. The formulations may include the oil nutrient D-limonene, a natural compound found in lemon and orange peels. The amount of D-Limonene included may be, for example, less than 10% by weight of the formulation. In the alternative, a terpene or terpene blend can be used in place of D-Limonene. Polyoxyl 40 hydrogenated castor oil is sometimes used as a non-ionic oil-in-water solubilizer and/or emulsifying agent. It is almost tasteless, particularly suitable for fat-soluble vitamins, and is widely used in self-emulsifying drug delivery systems or microemulsifying systems. Polyethylene glycol 300 (PEG-300) is a clear, colorless liquid made from sugar cane waste is soluble in water.

### Example Formulation 1

Example Formulation 1 may take the form of an oral spray. Phospholipids PC and PH may comprise 10% to 20% of the formulation by weight, with PC comprising greater than 75% of the mix between PC and PH. D-limonene may comprise less than 5% of the formulation, Polyoxyl 40 Hydrogenated Castor Oil may comprise 10% to 20% of the formulation; MCT Oil may comprises 10% to 20% of the formulation, and THC may comprise less than 3% of the formulation. The balance of the formulation may comprise PEG-300. An example embodiment of Example Formulation 1 is set forth below in Table 1.

TABLE 1

| | |
|---|---|
| PC 85% PH15% | 14.0% |
| D-limonene | 2.0% |
| Polyoxyl 40 Hydrogenated Castor Oil | 12.0% |
| MCT Oil | 16.0% |
| PEG-300 | 55.0% |
| THC | 1.0% |

When 5 mg of THC is ingested as part of Formulation 1, it may take approximately 20 minutes before onset of the drug effects. The peak may be felt in one hour, and the duration may be approximately three hours.

### Example Formulation 2

Example Formulation 2 may be a solid at room temperature, and may have a consistency appropriate for a capsule. Phospholipids PC and PH may comprise 15% to 25% of the formulation by weight, with PC comprising greater than 80% of the mix between PC and PH. D-limonene may comprise 5% to 10% of the formulation, and THC may comprise less than 3% of the formulation. The balance of the formulation may comprise Polyoxyl 40 Hydrogenated Castor Oil. An example embodiment of Example Formulation 2 is set forth below in Table 2.

TABLE 2

| | |
|---|---|
| PC 90% PH 10% | 21.4% |
| D-limonene | 7.1% |
| Polyoxyl 40 Hydrogenated Castor Oil | 70.5% |
| THC | 1.0% |

When a 5 mg THC capsule is ingested as part of Formulation 2, it may take approximately 15 minutes before onset of the drug effects, with a peak effect at approximately 45 minutes. The duration may be approximately three hours.

### Example Formulation 3

Example Formulation 3 may be a solid at room temperature, and may have a consistency appropriate for a capsule. Phospholipids PC and PH may comprise 15% to 25% of the formulation by weight, with PC comprising greater than 65% to 85% of the mix between PC and PH. D-limonene may comprise 5% to 10% of the formulation, and THC may comprise less than 3% of the formulation. The balance of the formulation may comprise Polyoxyl 40 Hydrogenated Castor Oil. An example embodiment of Example Formulation 3 is set forth below in Table 3.

TABLE 3

| | |
|---|---|
| PC 75% PH25% | 21.4% |
| D-limonene | 7.1% |
| Polyoxyl 40 Hydrogenated Castor Oil | 70.5% |
| THC | 1.0% |

When a 5 mg THC capsule is ingested as part of Formulation 3, it may take approximately 30 minutes before onset of the drug effects, with a peak effect at approximately 1.5 hours. The duration may be approximately four hours.

### Example Formulation 4

Example Formulation 4 may have the consistency of ground sugar. It can be added to any baked good, drink, or other edible product. Phospholipid PC may comprise 5% to 15% of the formulation by weight. Ethanol and THC may each comprise less than 3% of the formulation. Corn syrup may comprise 20% to 30% of the formulation. The balance of the formulation may comprise cane sugar. An example embodiment of Example Formulation 4 is set forth below in Table 4.

TABLE 4

| | |
|---|---|
| PC 100% | 5.7% |
| Ethanol | 1.4% |
| Cane Sugar | 67.1% |
| Corn Syrup | 24.8% |
| THC | 1.0% |

In its raw form, Example Formulation 4 may take approximately five to ten minutes for the onset of drug effects, and may last approximately 2 hours.

### Example Formulation 5

Example Formulation 5 may be a solid at room temperature, and may have a consistency appropriate for a capsule. Beeswax may comprise 5% to 15% of the formulation by weight, and THC may comprise less than 3% of the formulation. The balance of the formulation may comprise MCT Oil. An example embodiment of Example Formulation 5 is set forth below in Table 5.

TABLE 5

| | |
|---|---|
| MCT Oil | 89.0% |
| Beeswax | 10.0% |
| THC | 1.0% |

When a 5 mg THC capsule formed according to Example Formulation 5 is ingested, onset of the drug effects may not occur for approximately four hours, and no obvious peak may be felt. The duration may be approximately 6 hours. This timing would be suitable for a sustained release.

### Example Formulation 6

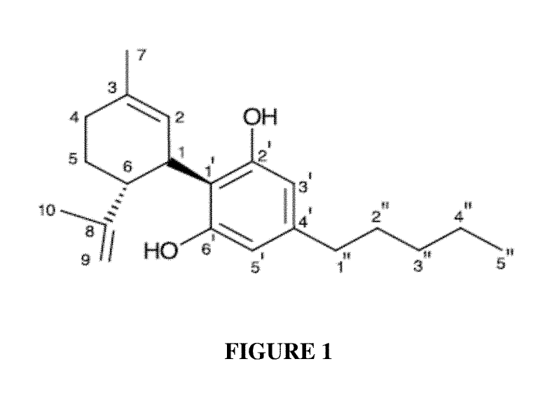

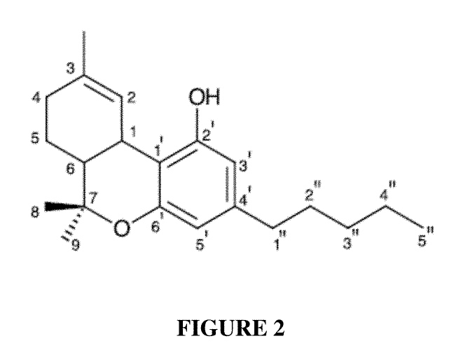

Example Formulation 6 may take the form of a hard chewy, fruit jelly or a peppermint patty. A lecithin emulsifier enriched with PC and/or PH may comprise 5% to 10% of the formulation by weight. One example of an emulsifier is Phospholipon™, manufactured by the American Lechtin Company. Lecithins are fatty substances occurring in animal and plant tissues. Lecithins are both hydrophilic and lipophilic, and are used for smoothing food textures, emulsifying, homogenizing liquid mixtures, and repelling sticking materials. In one example, the lecithin may be enriched with phospholipids comprising 50% PC and 50% PH. As another example, the lecithin may be enriched with phospholipids comprising 85% PC and 15% PH. Vitamin E may comprise 5% to 10% of the formulation by weight, Lemon Oil may comprise up to 1% of the formulation by weight, and Ethanol may comprise 5% to 10% of the formulation by weight. The balance of the formulation may comprise Glycerin. THC and/or Cannabidiol (CBD), a naturally occurring cannabinoid, may be added to the formulation. For example, 10 mg of THC may be included. FIG. 1 depicts the chemical structure of CBD, and FIG. 2 depicts the chemical structure of THC. Also, one mole of lecithin emulsifier, and as high as 20 or 25 moles of lecithin emulsifier, may be included for each mole of included cannabinoids. An example embodiment of Example Formulation 6 is set forth below in Table 6.

TABLE 6

| | |
|---|---|
| Lecithin fraction enriched with at least 85% PC | 8.0% |
| Vitamin E | 8.0% |
| Lemon Oil | 0.4% |
| Glycerin | 75.3% |
| Ethanol | 8.3% |

### Example Formulation 7

Example Formulation 7 may take the form of a gel-like formulation which may be used in a gel capsule or a gel-like edible. A lecithin emulsifier enriched with PC and/or PH may comprise up to 50% of the formulation by weight. The mix of phospholipids may be at least 50% PC and 50% PH. As another example, the mix of phospholipids may be at least 85% PC and 15% PH. Lemon Oil may comprise up to 3% of the formulation by weight. The balance of the formulation may comprise Polyoxyl 40 Hydrogenated Castor Oil. THC and/or CBD may be added to the formulation. For example, 10 mg of THC may be included. Also, one mole of lecithin emulsifier, and as high as 20 or 25 moles of lecithin emulsifier may be included for each mole of included cannabinoids. An example embodiment of Example Formulation 7 is set forth below in Table 7.

TABLE 7

| | |
|---|---|
| Lecithin fraction enriched with at least 85% PC | 49.5% |
| Polyoxyl 40 Hydrogenated Castor Oil | 49.5% |
| Lemon Oil | 1.0% |

### Example Formulation 8

Example Formulation 8 may take the form of a cannabis-infused sugar. A lecithin emulsifier enriched with PC and/or PH may comprise 5% to 15% of the formulation by weight. The mix of phospholipids may be at least 50% PC and the remainder PH. As another example, the mix of phospholipids may be 85% PC and 15% PH. Corn syrup may comprise up to 25% of the formulation by weight. The balance of the formulation may comprise Sugar. THC and/or CBD may be added to the formulation. For example, 10 mg of THC may be included. An example embodiment of Example Formulation 8 is set forth below in Table 8.

TABLE 8

| | |
|---|---|
| Lecithin fraction enriched with at least 85% PC | 10.0% |
| Sugar | 65.0% |
| Corn Syrup | 25.0% |

### Example Formulation 9

Example Formulation 9 may take the form of a powder. The powder may be added to a liquid for consumption. The formulation may comprise 15 to 25 grams or 1.7% to 3% by weight of lecithin emulsifier enriched with PC and/or PH. The mix of phospholipids may be at least 50% PC and the remainder PH. As another example, the mix of phospholipids may be 85% PC and 15% PH. Formulation 9 may further comprise 15 to 25 grams or 1.7% to 3% by weight of Vitamin E TPGS, 75 to 150 grams or 8.7% to 17.4% by weight of Ethanol, 400 to 500 grams or 46% to 58.1% of cane sugar, 150 to 200 grams or 17% to 24% by weight of corn syrup, up to 100 grams or up to 12% by weight of water, and 15 to 25 grams or 1.7% to 3% by weight of THC. An example embodiment of Example Formulation 9 is set forth below in Table 9.

TABLE 9

| | |
|---|---|
| Lecithin fraction enriched with at least 85% PC | 2.3% |
| Vitamin E TPGS | 2.3% |
| Ethanol | 11.6% |
| Cane Sugar | 55.0% |
| Corn Syrup | 20.6% |
| Water | 5.9% |
| THC | 2.3% |

### Example Formulation 10

Example Formulation 10 may take the form of a water-soluble mix. A lecithin emulsifier enriched with PC and/or PH, such as Phospholipon™, may comprise 20% to 30% of the formulation by weight. In one example, the lecithin may be enriched with phospholipids comprising 50% PC and 50% PH. As another example, the lecithin may be enriched with phospholipids comprising 85% PC and 15% PH. Vitamin E may comprise 20% to 30% of the formulation by weight, Polysorbate 80 may comprise up to 15% of the formulation by weight, and a cannabinoid (e.g., THC and/or Cannabidiol (CBD)) may comprise 3% to 10% of the formulation by weight. The balance of the formulation may comprise Ethanol. An example embodiment of Example Formulation 10 is set forth below in Table 10.

TABLE 10

| | |
|---|---|
| Lecithin fraction enriched with at least 85% PC | 26.0% |
| Vitamin E TPGS | 26.0% |
| Polysorbate 80 | 12.0% |
| Cannabinoids | 5.0% |
| Ethanol | 31.0% |

While the invention has been described with reference to various embodiments, which embodiments have been set forth in considerable detail for the purposes of making a complete disclosure of the invention, such embodiments are merely exemplary and are not intended to be limiting or represent an exhaustive enumeration of all aspects of the invention. Further, it will be apparent to those of skill in the art that numerous changes may be made in such details without departing from the spirit and the principles of the invention. It should be appreciated that the invention is capable of being embodied in other forms without departing from its essential characteristics.

What is claimed is:

1. A composition for administering cannabinoid, comprising:
phospholipids;
D-limonene;
polyoxyl 40 hydrogenated castor oil; and
delta-9-tetrahydrocannabinol.

2. The composition of claim 1, wherein the composition comprises the following percentages by weight:
at least 14.0% phospholipids;
at least 2.0% D-limonene;
at least 12.0% polyoxyl 40 hydrogenated castor oil; and
at least 1.0% delta-9-tetrahydrocannabinol.

3. The composition of claim 2, wherein the phospholipids comprise phosphatidylcholine and phosphatidylethanolamine.

4. The composition of claim 3, wherein the phospholipids are comprised of at least 85% phosphatidylcholine.

5. The composition of claim 3 further comprising approximately 16% by weight of medium chain triglyceride oil

6. The composition of claim 5 further comprising approximately 55% by weight of polyethylene glycol 300.

7. The composition of claim 1, wherein the composition comprises approximately the following percentages by weight:
approximately 21.0% phospholipids;
approximately 7.0% D-limonene;
approximately 70.0% polyoxyl 40 hydrogenated castor oil; and
approximately 1.0% delta-9-tetrahydrocannabinol.

8. The composition of claim 7, wherein the phospholipids comprise phosphatidylcholine and phosphatidylethanolamine.

9. The composition of claim 9, wherein the phospholipids are comprised of at least 75% phosphatidylcholine.

10. The composition of claim 9, wherein the phospholipids are comprised of at least 90% phosphatidylcholine.

11. A composition for administering cannabinoid, comprising approximately the following percentages by weight:
8.0% lecithin fraction enriched with phospholipids;
8.0% vitamin E;
0.4% lemon oil;
75.3% glycerin; and
8.3% ethanol.

12. The composition of claim 11 wherein the phospholipids are comprised of at least 50% Phosphatidylcholine.

13. The composition of claim 12 further comprising 10 milligrams of delta-9-tetrahydrocannabinol.

14. The composition of claim 13, wherein the composition comprises 20 moles of lecithin fraction enriched with at least 85% phosphatidylcholine for each mole of delta-9-tetrahydrocannabinol.

15. The composition of claim 11 wherein the phospholipids are comprised of at least 85% phosphatidylcholine.

16. The composition of claim 15 further comprising 10 milligrams of delta-9-tetrahydrocannabinol.

17. The composition of claim 16, wherein the composition comprises 20 moles of lecithin fraction enriched with at least 85% phosphatidylcholine for each mole of delta-9-tetrahydrocannabinol.

18. A composition for administering cannabinoid, comprising approximately the following percentages by weight:
26% lecithin fraction enriched with phospholipids;
26% vitamin E TPGS;
12% polysorbate 80;
5% cannabinoids; and
31% ethanol.

19. The composition of claim 19 wherein the phospholipids are comprised of at least 50% phosphatidylcholine.

20. The composition of claim 19 wherein the phospholipids are comprised of at least 85% phosphatidylcholine.
